(19) United States
(12) Patent Application Publication
(10) Pub. No.: US 2023/0201401 A1
(43) Pub. Date: Jun. 29, 2023

(54) FILTER INSERT AND DEVICE FOR STERILIZATION AND/OR DISINFECTION THEREOF

(71) Applicant: CESKE VYSOKE UCENI TECHNICKE V PRAZE, 16000 Praha 6, Dejvice (CZ)

(72) Inventors: Lukas VOJTECH, Nahorany (CZ); Marek NERUDA, Hradec Kralove (CZ); Pavel HNYK, Praha (CZ); Jiri VODRAZKA, Praha (CZ)

(73) Assignee: CESKE VYSOKE UCENI TECHNICKE V PRAZE, 16000 Praha 6, Dejvice (CZ)

(21) Appl. No.: 17/927,821

(22) PCT Filed: May 28, 2021
(86) PCT No.: PCT/IB2021/054677

§ 371 Date: Nov. 25, 2022

(30) Foreign Application Priority Data
May 28, 2020 (CZ) PV 2020-305

(51) Int. Cl. A61L 9/014; B01D 39/16; A61L 9/16; A61L 2/26; A61L 2/02; A61L 2/04

(52) U.S. Cl. CPC A61L 9/014; B01D 39/1623; A61L 9/16; A61L 2/26; A61L 2/022; A61L 2/04; B01D 2239/0241; B01D 2239/065; B01D 2239/0442; B01D 2239/025; A61L 2209/14; A61L 2209/22; A62B 23/02

(57) ABSTRACT

A filter insert for filtration of liquids and/or gases includes at least one filter layer containing at least one electro-conductive layer to resist heating of the filter insert. Further, it relates to a device for sterilization and/or disinfection of the filter insert, in particular, the device for sterilization and/or disinfection of the filter insert for filtration of liquids and/or gases, according to any one of the preceding claims, including at least one filter layer, wherein it includes the electric power supply connectable by at least two conductive contacts to the electro-conductive layer of the filter insert to resist heating of it.

### BACKGROUND AND SUMMARY

The invention relates to a filter insert, in particular, a filter insert for filtration of liquids and/or gases and a device for sterilization and/or disinfection thereof.

Filtration materials are an integral part of human and environmental protection systems against dangerous and undesirable influences of technologies or organisms in the filtration of liquids and gases. A special category is a biological protection, especially protection against microorganisms such as viruses and bacteria contained in aerosols. In these cases, filters in the form of face mask or filters of breathing masks or half masks are used to protect the human and environment. These filters employ various planar textile structures made of various composites, wherein layers with planar nanostructures are employed as well. Such made filters are either disposable or allow sterilization or disinfection using various reagents, equipment, instruments, or auxiliary technologies.

So far, several principal procedures are used for general processes of sterilization or disinfection, based mainly on physical or chemical methods. The physical methods include solutions using steam known from CN203252939 or hot air as known from US2020061230 and CN207640703, hot water according to JP2003092955, ultraviolet radiation according to JP2018181476 or low-temperature plasma according to CN105173249, ultrasound or a combination of several methods described herein according to CN101722160. In addition, known physical methods of sterilization or disinfection employ various types of ionizing or non-ionizing radiation, such as the use of high-frequency electromagnetic fields according to CN2102009. The chemical methods include the use of additional layers of chemicals according to RU2461675, the use of ozone according to JP2004105423 or electrolytes according to JPH02111371 and CN2087077.

The above-mentioned state of the art shows that the previously known processes of sterilization or disinfection for filters are either technologically demanding or difficult to perform due to the need to handle chemicals and, in addition, they often require complex logistical support or are not suitable for the selected filter types at all.

It is desirable to provide a filter insert design enabling easy and highly efficient sterilization and/or disinfection and provide a device design enabling easy and cheap sterilization and/or disinfection of the filter insert.

The said disadvantages are largely eliminated by a filter insert, specifically a filter insert for the filtration of liquids and/or gases, which comprises at least one filter layer according to an aspect of the present invention, characterized in that it comprises at least one electro-conductive layer to resist heating of the filtration insert. This design's main advantage is that the filter insert, or even the filter face mask directly, can be very easily disinfected and sterilized by resistance heating. Such formed electro-conductive layer may complete the existing designs of various textile, paper, ceramic or other general filters, thus increasing utility value thereof.

Preferably, at least a part of the electro-conductive layer is ready for contact with the electrodes. The advantage consists in that the electrical connection is direct, wherein, on the one hand, there are no losses, and on the other hand and most importantly, the heating temperature of the filter insert can be precisely set as a result.

Further, it is preferable when the electro-conductive layer is formed by an electro-conductive textile, which is a copper-plated textile in preferred embodiments, or the textile is a carbon fabric. The main advantage consists in a simple design. The parameters of the current flow can be precisely set, and, as a result, the heating temperature of the filter insert can also be precisely set. The electro-conductive layer can also be formed, for example, by sewing a simple textile layer by using conductive fibres. Preferably, other galvanically plated fabrics or mixed fabrics with electro-conductive fibres are possible to be used. The electro-conductive layer can also be implemented via a metal grid or mesh, by printing an electro-conductive structure on a textile substrate, by inweaving electro-conductive structures, via an electro-conductive fabric, and the like.

Further, the electro-conductive layer is preferred to contain a component having virucidal effects. Most preferably, the electro-conductive layer may be formed by electro-conductive materials themselves exhibiting, for example, virucidal effects, such as silver and copper. The advantage consists in an increase in the resistance of the filter insert to viruses during the normal use thereof.

More preferable, the filter layer is made of nano-textile. The advantage consists of the nanotextile's excellent filtration characteristics, especially when capturing microscopic particles, such as viruses. Alternatively, the filter layer may be made of textile, paper, ceramic or another generally known filtration material, the electro-conductive layer increasing the utility value thereof.

Further, the filter insert preferably comprises at least one protective layer, wherein, the most preferably, the protective layer is made of a non-woven textile. The advantage consists in that the functional layers of the filter insert are simply protected against mechanical damage.

Further, the filter insert as a whole is preferably resistant to a temperature of at least 80° C. The advantage consists in that the filter insert can withstand a temperature reliably destroying virtually all viruses and bacteria.

According to a preferred embodiment, the electro-conductive layer may advantageously be used as a filter layer as well. The advantage consists in a simpler and lighter design of the entire filter insert.

The main advantage of the filter insert, according to an aspect of the invention, consists in that it can be easily, cheaply and repeatedly regenerated; more precisely, it can be disinfected or sterilized by heating. At the same time, the filter insert is thus possible to be dried since the passing electric current of a sufficient level heats the filter structure to the required temperature, and the structure is therefore directly disinfected or sterilized through this process for the required time.

The above drawbacks are largely overcome by a device for sterilization and/or disinfection of the filter insert, in particular, a device for sterilization and/or disinfection of the above-mentioned filter insert for filtering liquids and/or gases, which comprises at least one filter layer according to an aspect of this invention, characterized in that it comprises a source of electric current connectable by at least two conductive contacts with the electro-conductive layer of the filter insert to resist heating of it. The advantage consists in that such a device is quite simple and fully operational, wherein it is also easily portable in most of the embodiments thereof.

The electric power supply is more preferably a low-voltage electric power supply of up to 50 V. The advantage consists in that such a device is both very safe and, at the same time, easily connectable to most available power sources; for example, it is easy to be connected to electric power sources available in cars.

The conductive contacts are preferably conductively connected to busbars where the filter insert is conductively placed. The advantage consists in that the busbars with different types of filter inserts are possible in standardized housings, which may be arranged directly in the filtration devices, for example.

The conductive contacts are preferably conductively connected to the busbars via magnetic contacts arranged on a supply connector connected to the electric power supply. The advantage consists in easy connection and disconnection of the filter insert for heat treatment.

From a practical point of view, the busbars are more preferably placed in the filtration element body simultaneously. The advantage consists in that the filter insert may be easily and quickly treated without disassembling it, while the surrounding thereof is heat-treated at the same time.

Furthermore, the filter insert is very preferably fastened to the busbar by clamping rotation. Most preferably, the filter insert is fastened to the busbar by clamping rotation of the electro-conductive layer thereof. The advantage consists in that contacting the electro-conductive layer with the busbar by rotating it on the busbar allows a continuous decrease in the electrical conductivity of the electro-conductive layer, from the highly electro-conductive busbar to the resistance electro-conductive layer. With such solution, there cannot be places with a step-change in the electrical resistance between the busbar and the electro-conductive layer, which could otherwise cause overheating or even burning of the electro-conductive layer.

According to another preferred embodiment, the conductive contacts are arranged in a sterilization means, wherein the sterilization means preferably comprises a pressure frame to press the electro-conductive layer of the filter insert onto the conductive contacts. The advantage consists in that any filter insert of a specific shape can be treated.

Further, the pressure frame is preferably pivoted in the sterilization means through a joint. The advantage consists in that the filter insert can be easily pressed onto the conductive contacts.

Then, the pressure frame is preferably held to press the electro-conductive layer of the filter insert onto the conductive contacts by at least one locking element.

The main advantage of the device, according to an aspect of the invention, consists in that it makes it possible to carry out easy regeneration, more precisely sterilization or disinfection of the filter insert, namely through heating by passing electric current while drying the filter insert. Such a solution allows the filter to be used repeatedly for the filtration of liquids and gases, particularly for respiratory protection or the protection against the spread of airborne diseases. The technical solution of the filter insert and the device intended for the sterilization and/or disinfection thereof increases the possibility of protection of a larger number of inhabitants or endangered groups against airborne viral diseases with the same amount of available filtration material as a result of repeated use of the filter insert. A great advantage consists in that radiant heat is used for sterilization and/or disinfection, thermally disinfecting or sterilizing the surroundings thereof in addition, wherein such surrounding may be directly the filter body containing the filter insert. In addition, this method of disinfection or sterilization by internal heating of the filter insert generates dry heat, which is often recommended for these processes. The heating is maximum inside the electro-conductive layer, as the surrounding environment cools the surface thereof. As a result, the filter insert is highly efficiently heated and dried due to the evaporation of liquids or moisture from the entire volume of material. In addition, this solution speeds up the overall disinfection or sterilization process due to the rapid drying.

### BRIEF DESCRIPTION OF DRAWINGS

The invention will be explained in detail referring to the accompanying drawings, wherein

FIG. 1 shows a perspective view of a device for sterilizing and/or disinfecting a filter insert which is placed directly in the filtration element body via busbars;

FIG. 2 shows a perspective view of the busbar, onto which the filter insert end is rolled;

FIG. 3 shows a detailed cross-sectional view of the busbar, onto which the filter insert end is rolled;

FIG. 4 shows a perspective view of the filtration element, where the filter insert is arranged;

FIG. 5 shows a perspective view of the sterilization or/and disinfection device, in sterilization means of which, there is the filter insert arranged on the conductive contacts;

FIG. 6 shows in a perspective view a detail of the filter insert on the conductive contacts; and

FIG. 7 shows a detailed cross-sectional view of the filter insert placement on the conductive contacts.

### DETAILED DESCRIPTION

### EXAMPLE 1

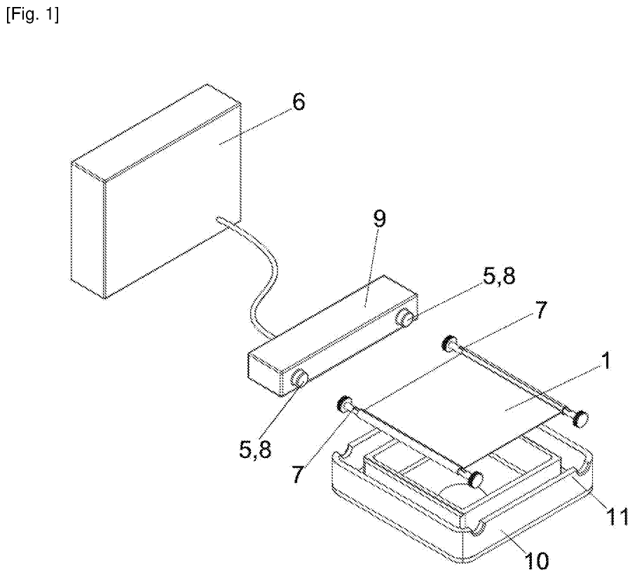

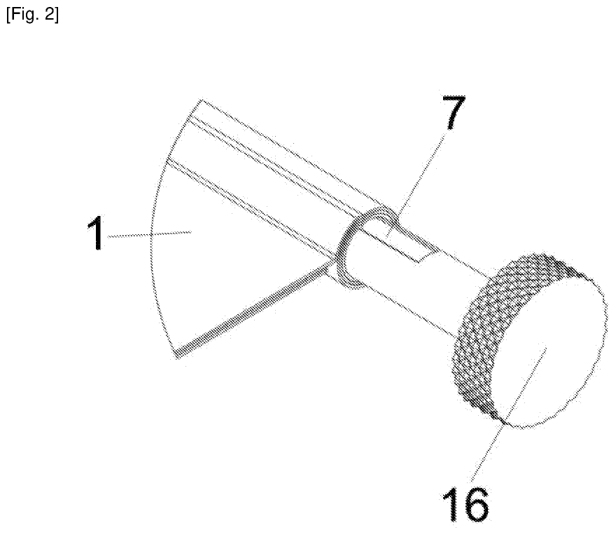

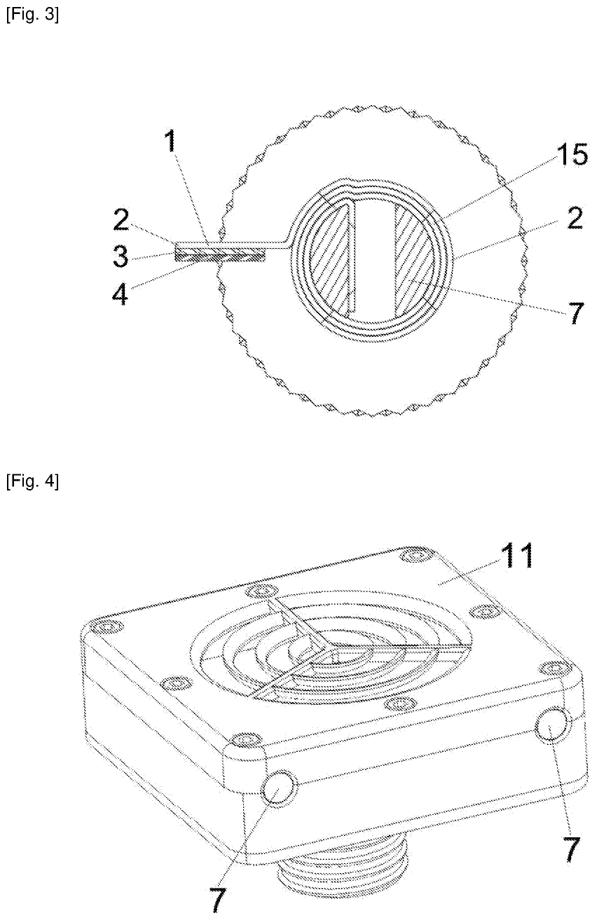

The filter insert 1 according to FIG. 1, FIG. 2, and FIG. 3 for filtration of liquids and/or gases has a rectangular shape, comprising a filter layer 3, where an electro-conductive layer 2 is arranged on one side to resist heating of the filter insert 1, and a protective layer 4 is arranged on the other side.

The electro-conductive layer 2 is made of an electro-conductive textile, which is a carbon fabric.

The electro-conductive layer 2 contains a component featuring virucidal effects, consisting of or comprising silver or copper nanoparticles.

Filter layer 3 consists of or comprises a nanotextile made of PVDF (polyvinylidene fluoride) to increase the filtering effect against viruses spread by airborne infection.

The protective layer 4 is made of a nonwoven textile.

The filter insert 1 as a whole is resistant to a temperature of 80° C.

Alternatively, the electro-conductive layer 2 may be used as the filter layer 3 as well.

The device, according to FIG. 1, FIG. 2, FIG. 3 for sterilization and/or disinfection of the filter insert 1 for filtration of liquids and/or gases, comprises an electric power supply 6 connectable by two conductive contacts 5 to the electro-conductive layer 2 of the filter insert 1 to resist heating of it.

The electric power supply 6 is a direct-current source.

The electric power supply 6 is a low-voltage electric power supply of up to 12 V.

The conductive contacts 5 are conductively connected to the busbars 7 via magnetic contacts 8 arranged on a supply connector 9 connected to the electric power supply 6.

At the ends thereof, the busbars 7 are provided with knobs 16. The busbars 7 are housed in the body 10 of the filtration element 11 according to FIG. 4.

The filter insert 1 is fastened to the busbar 7 by rotary clamping 15 of the electro-conductive layer 2 thereof.

The filtration element 11 is a part of a respiratory half mask. The filter insert 1 of the filtration element 11 is possible to be disinfected both directly, as a component of the respiratory half mask, and separately, after it has been removed from the filtration element 11. After connecting to the electric power supply 6, the filter element 1 is heated to a temperature of 80° C. within 60 seconds as a result of the passing electric current. The disinfection process takes place for one hour at a temperature of 80° C. After disconnecting the filter insert 1 from the electric power supply 6, the filter insert 1 cools down spontaneously, and the filtration element 11 is thus disinfected and ready for next use.

### EXAMPLE 2

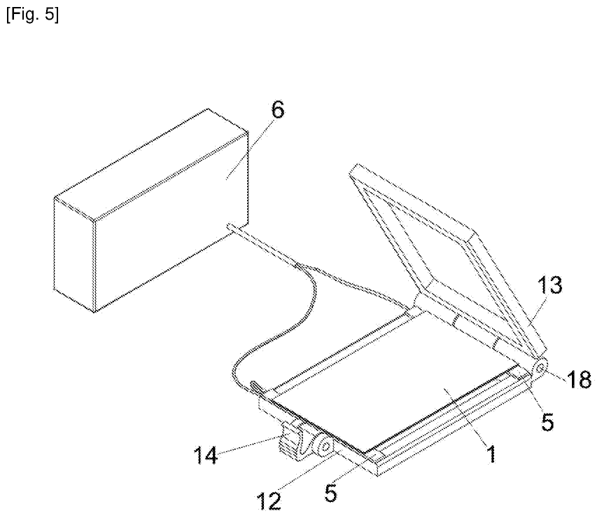

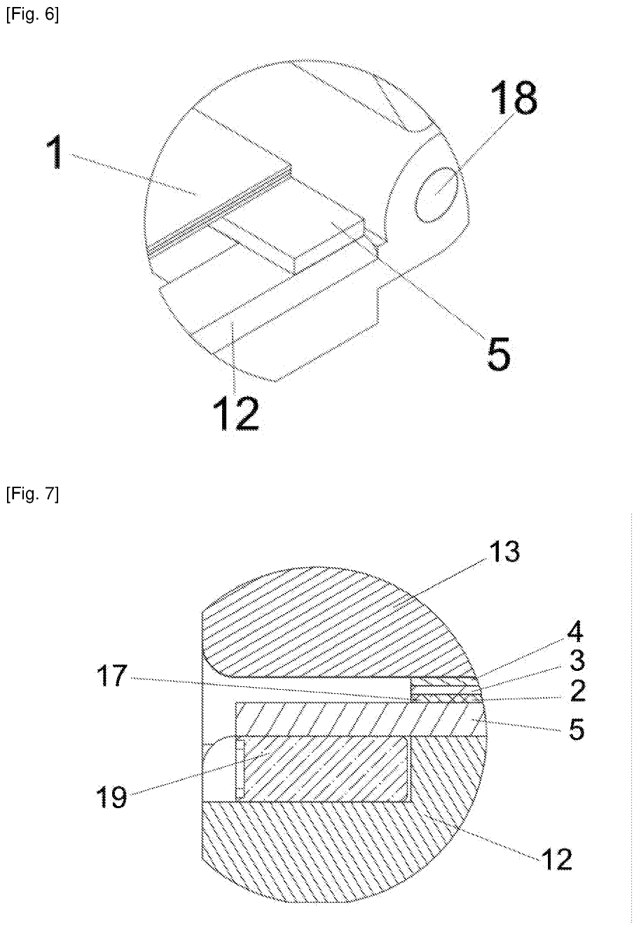

The filter insert 1, according to FIG. 5, FIG. 6, and FIG. 7 for filtration of liquids and/or gases, has a rectangular shape, comprising a filter layer 3, where an electro-conductive layer 2 is arranged on one side to resist heating of the filter insert 1, and a protective layer 4 is arranged on the other side.

Part 17 of the electro-conductive layer 2 is ready to contact the conductive contacts 5.

The electro-conductive layer 2 is made of an electro-conductive textile, which is a copper-plated textile.

Filter layer 3 consists of or comprises a nanotextile made of PVDF (polyvinylidene fluoride).

The protective layer 4 is made of a nonwoven textile.

The filter insert is resistant to a temperature of at least 80° C.

The device, according to FIG. 5, FIG. 6, FIG. 7 for sterilization and/or disinfection of the filter insert 1 for filtration of liquids and/or gases, comprises an electric power supply 6 connectable by two conductive contacts 5 to the electro-conductive layer 2 of the filter insert 1 to resist heating of it.

The electric power supply 6 is a direct-current source.

The electric power supply 6 is a low-voltage electric power supply of up to 48 V.

The conductive contacts 5 are arranged in the sterilization means 12. The conductive contacts 5 directly contact the electro-conductive layer 2 of the filter insert 1.

The sterilization means 12 comprises a pressure frame 13 to press the electro-conductive layer 2 of the filter insert 1 onto the conductive contacts 5.

The pressure frame 13 is rotatably mounted in the sterilization means 12 by a joint 18.

The pressure frame 13 is held to press the electro-conductive layer 2 of the filter insert 1 onto the conductive contacts 5 by the locking element 14.

The filter insert 1 is arranged in a pocket of a textile face mask. After removal thereof from the textile face mask, the filter insert 1 is placed into the sterilization means 12 and parts 17 of the electro-conductive layer 2 thereof are pressed by the pressure frame 13 to contact the conductive contacts 5. After connecting to the electric power supply 6, the filter insert 1 is heated to 120° C. within 60 seconds due to the passing electric current. The disinfection process takes place for 90 minutes at the temperature of 120° C. The passing electric current generates heat by means of resistance losses in the galvanically copper-plated textile, causing an increase in temperature inside and on the surface of the entire composite. The maximum temperature, which the material of the filter insert 1 can withstand repeatedly and without degradations, is controlled by the shown reversible thermally-controlled disconnecting switch 19 connected in series to the electrical circuit of the heated filter insert 1. The filter insert 1 heat-sterilized in this way is removed from the member after cooling; and it is ready for re-use. After disconnection from the electric power supply 6, the filter insert 1 cools down spontaneously and is thus disinfected and ready for further use.

The filter insert, according to an aspect of the invention, is possible to be used to filter liquids and/or gases with simple subsequent sterilization and/or disinfection on the device according to an aspect of the invention to eliminate various types of captured pathogens, such as bacteria and viruses.

### REFERENCE SIGNS LIST

1 Filter insert

2 Electro-conductive layer

3 Filter layer

4 Protective layer

5 Conductive contact

6 Electric power supply

7 Busbar

8 Magnetic contact

9 Supply connector

10 Filtration element body

11 Filtration element

12 Sterilization means

13 Pressure frame

14 Locking element

15 Clamping rotation

16 Knob

17 Part of the electro-conductive layer

18 Joint

19 Reversible thermally-controlled disconnecting switch

1. A filter insert for the filtration of liquids and/or gases, comprising at least one filter layer, wherein the filter insert contains at least one electro-conductive layer to resist heating of the filter insert.

2. The filter insert according to claim 1, wherein at least a part of the electro-conductive layer is ready for contact with the electrodes.

3. The filter insert according to claim 1, wherein the electro-conductive layer is made of an electro-conductive textile.

4. The filter insert according to claim 3, wherein the electro-conductive textile is a copper-plated textile.

5. The filter insert according to claim 3, wherein the electro-conductive textile is a carbon fabric.

6. The filter insert according to claim 1, wherein the electro-conductive layer contains a component with virucidal effects.

7. The filter insert according to claim 1, wherein the filter layer is made of a nano-textile.

8. The filter insert according to claim 1, wherein it further comprises at least one protective layer.

9. The filter insert according to claim 8, wherein the protective layer is made of a non-woven textile.

10. The filter insert according to claim 1, wherein it is resistant to a temperature of at least 80° C.

11. The filter insert according to claim 1, wherein the electro-conductive layer is simultaneously used as filter layer of the filter.

12. A device for the sterilization and/or disinfection of the filter inserts, according to claim 1, comprising at least one filter layer, wherein the device comprises an electric power supply connectable by at least two conductive contacts to an electro-conductive layer of the filter insert to resist heating of it.

13. The device according to claim 12, wherein the electric power supply is a low-voltage electric power supply of up to 50 V.

14. The device according to claim 12, wherein the conductive contacts are conductively connected to busbars where the filter insert is conductively placed.

15. The device according to claim 14, wherein the conductive contacts are conductively connected to the busbars via magnetic contacts arranged on a supply connector connected to the electric power supply.

16. The device according to claim 14, wherein the busbars are simultaneously placed within a body of a filtration element.

17. The device according to claim 1, wherein the filter insert is fastened to the busbar by rotary clamping.

18. The device according to claim 17, wherein the filter insert is fastened to the busbar by rotary clamping of the electro-conductive layer thereof.

19. The device according to claim 12, wherein the conductive contacts are arranged in a sterilization means.

20. The device according to claim 19, wherein the sterilization means comprises a pressure frame to press the electro-conductive layer of the filter insert onto the conductive contacts.

21. The device according to claim 20, wherein the pressure frame is, by a joint, rotatably mounted in the sterilization means.

22. The device according to claim 20, wherein the pressure frame is held to press the electro-conductive layer of the filter insert onto the conductive contacts with at least one locking element.
